(19) United States
(12) Patent Application Publication
(10) Pub. No.: US 2007/0163603 A1
(43) Pub. Date: Jul. 19, 2007

(54) Tongue retention device and method of use

(76) Inventor: Val J. Sikora, Fort Myers, FL (US)

Correspondence Address: EDWARD M. LIVINGSTON, PA, 963 TRAIL TERRACE DRIVE, NAPLES, FL, 34103, US

(21) Appl. No.: 11/334,216

(22) Filed: Jan. 18, 2006

(51) Int. Cl. A61F 5/56

(52) U.S. Cl. 128/848

(57) ABSTRACT

A tongue retention device (1) for alleviating sleep apnea and other sleep maladies has a tooth-anchoring piece (12) that is positioned against the teeth (10) and a tab (2) having at least one aperture (3) for the acceptance of a tongue attachment device (9). To use the tongue retention device (1), a person places the anchoring piece (12) within his or her mouth. Then, he or she secures the attachment device (9), which may be tongue piercing or implant (13) located in the tongue (8), in the aperture (3) on the tab (2). Because the attachment device (9) is located in the tongue (8), the tongue (8) is maintained in a forward position such that it will not fall back into the throat of the wearer when the attachment device (9) is secured in the tab (2).

### BACKGROUND OF THE INVENTION

This invention relates to tongue retention devices, more particularly, a tongue retention device that maintains a tongue in a forward position to help alleviate sleep apnea and snoring.

Obstructed sleep apnea is a serious problem for a large amount of individuals wherein the soft palate muscles around the base of the tongue and the uvula relax when a person is asleep, thereby obstructing the person's airway. Because the airway is obstructed, the level of oxygen in the blood falls to dangerously low levels and causes hypoxia, a condition wherein there is a deficiency in the amount of oxygen reaching body tissues. In addition, the airway blockage also causes increased stress on the heart, elevated blood pressure and prevents individuals from entering REM sleep, the restful and restorative stage of sleep. In its most unobtrusive form, obstructed sleep apnea will cause a person to lose precious sleep and be tired the next day. In its most obtrusive form, obstructed sleep apnea could cause death as a person may block his or her airway and choke to death. Thus, the condition of obstructed sleep apnea is a very real and very serious medical condition which must be properly monitored and treated.

Currently, a person may use a continuous positive airway pressure (“CPAP”) machine to maintain the airway in an open position. Although the machine does prevent the airway from being blocked, it is not conducive for a restful night's sleep as the machine itself is very noisy. In addition, a person must wear a cumbersome air mask while sleeping, which is very uncomfortable.

In extreme cases, a person diagnosed and suffering from obstructed sleep apnea may undergo ENT surgery, also known as UPPP (uvuol—palatopharyngoplasty), where a surgeon removes excessive soft tissue in the back of the throat to relieve obstruction. Although the surgery does, in most cases, correct the problem, undergoing such a medical procedure is not only expensive, but also potentially life-threatening.

The most popular method of treatment for sleep apnea is surgery to remove uvula and/or the soft palate. Another new method is via the use of tongue retention devices that hold a person's tongue in a forward position while he or she is sleeping. Although there are numerous tongue retention devices that attempt to hold a person's tongue in a forward position during sleep so as to treat obstructed sleep apnea, the devices are defective in the fact that suction is used to maintain the tongue in the device. Therefore, although a person may fall asleep with the tongue in a forward position in the device, after a period of time, the tongue inevitably slips out of the device, falls back and obstructs the person's airway.

Thus, a need exists for a tongue retention device that permits a person to safely and easily maintain a person's tongue in a forward position so as to prevent blockage of the airway. The relevant prior art includes the following references:

| Patent No. | | |
|---|---|---|
| (U.S. unless | | |
| stated otherwise) | Inventor | Issue/Publication Date |
| 6,408,851 | Karell | Jun. 25, 2002 |
| 4,304,227 | Samelson | Dec. 8, 1981 |
| 2002/0144685 | Ivanovich et al. | Oct. 10, 2002 |
| 4,676,240 | Gardy | Jun. 30, 1987 |
| 5,988,171 | Sohn et al. | Nov. 23, 1999 |
| 2001/0027793 | Tielemans | Oct. 11, 2001 |
| 6,408,852 | Tielemans | Jun. 25, 2002 |
| 2005/0061334 | Krueger et al. | Mar. 24, 2005 |

### SUMMARY OF THE INVENTION

The primary object of the present invention is to provide a tongue retention device that maintains a person's tongue in a forward position while he or she is asleep.

A further object of the present invention is to provide a tongue retention device that is easy to use.

An even further object of the present invention is to provide a tongue retention device that is comfortable to wear.

Another object of the present invention is to provide a tongue retention device that is durable.

The present invention fulfills the above and other objects by providing a tongue retention device having an anchoring piece that is placed within a person's mouth and positioned such that it is against the teeth. The anchoring piece includes a tab having at least one aperture for the acceptance of an attachment device. To use the tongue retention device, a person positions the anchoring piece within his or her mouth. Then, he or she secures an attachment device, such as a tongue piercing or implant located in the tongue, in the aperture on the tab. Because the attachment device is located in the tongue, the tongue is maintained in a forward position such that it will not fall back into the throat of the wearer when the attachment device is secured to the tab.

The above and other objects, features and advantages of the present invention should become even more readily apparent to those skilled in the art upon a reading of the following detailed description in conjunction with the drawings wherein there is shown and described illustrative embodiments of the invention.

### BRIEF DESCRIPTION OF THE DRAWINGS

In the following detailed description, reference will be made to the attached drawings in which:

FIG. 1 is a perspective view of a tongue retention device of the present invention;

FIG. 2 is a frontal view of the embodiment of FIG. 1;

FIG. 3 is a side cut-away plan view of the tongue retention device of the present invention in use with a first embodiment of an attachment device;

FIG. 4 is a rear view of the present invention;

FIG. 5 is a perspective view of the present invention in use; and

FIG. 6 is a side view of a second embodiment of an attachment device for use in conjunction with the present invention.

### DESCRIPTION OF THE PREFERRED EMBODIMENTS

For purposes of describing the preferred embodiment, the terminology used in reference to the numbered components in the drawings is as follows:

1. tongue retention device, generally

2. tab

3. aperture

4. first wall

5. channel

6. vent

7. second wall

8. tongue

9. attachment device

10. teeth

11. lip

12. anchoring piece

13. implant

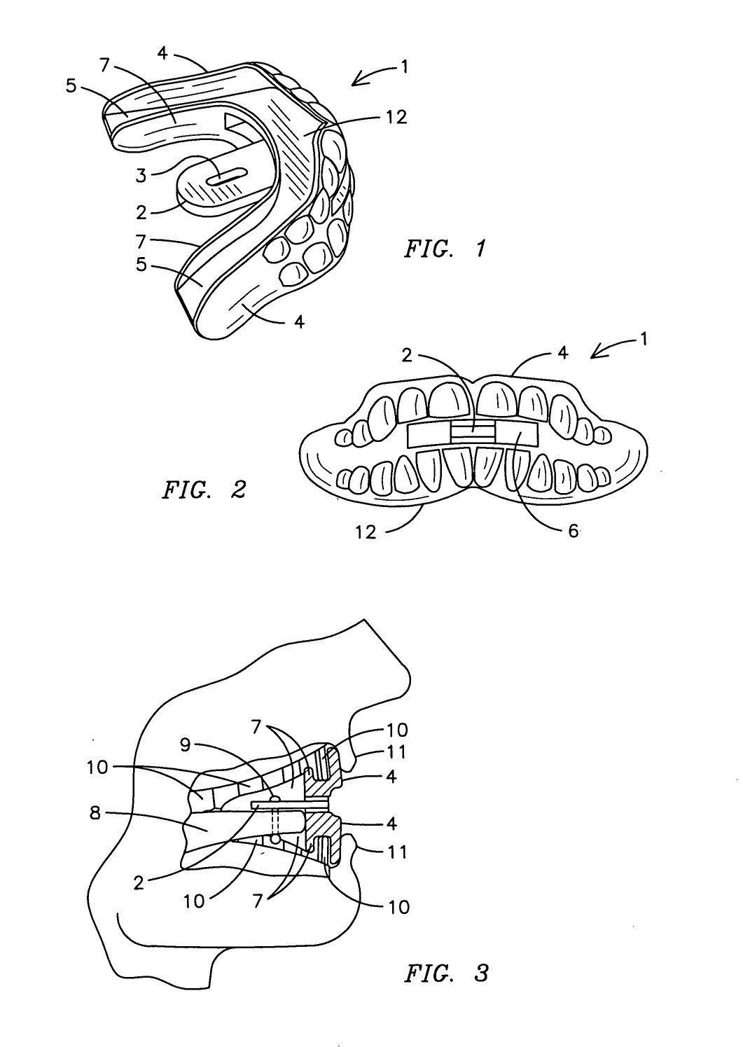

With reference to FIG. 1, a perspective view of a tongue retention device 1 of the present invention is shown. The tongue retention device 1 includes an anchoring piece 12 having a tab 2 secured thereto. The tab 2 includes at least one aperture 3 of a predetermined size. The at least one aperture 3 is predeterminedly-sized so as to accept an attachment device 9. The anchoring piece 12 includes a first wall 4 and a second wall 7 having a channel 5 located therebetween for the acceptance of a person's teeth. The tongue retention device 1 is preferably made of a rubber or soft plastic material so as to mold to and fit comfortably within a person's mouth. In addition, if the tongue retention device 1 is constructed of a rubber or soft plastic, the device 1 may also assist in the prevention of teeth grinding while sleeping. In addition, the tab 2 may be removably insertable into the tongue retention device 1 so as to permit a user to selectively secure his/her tongue in a predetermined position.

In FIG. 2, a frontal view of the embodiment of FIG. 1 is shown. A vent 6 is preferably located in a central area of the anchoring piece 12 so as to permit air flow through the anchoring piece 12.

FIG. 3 shows a side cross-sectional view of the tongue retention device 1 of the present invention in use with a first embodiment of an attachment device 9 located in a person's tongue 8. To use the present invention, a person secures the attachment device 9, which is preferably a tongue-piercing, such as a barbell or ball, within the aperture 3 located on the tab 2 and positions his or her teeth 10 within the channels 5 of the anchoring device 12. Because the tongue 8 is secured to the tongue retention device 1, the person is unable to move the tongue 8 a substantial amount of distance. When the person falls asleep and his or her tongue 8 relaxes, rather than falling backward into his or her throat, the tongue 8 remains in the same forward position, thereby permitting the person to breathe normally.

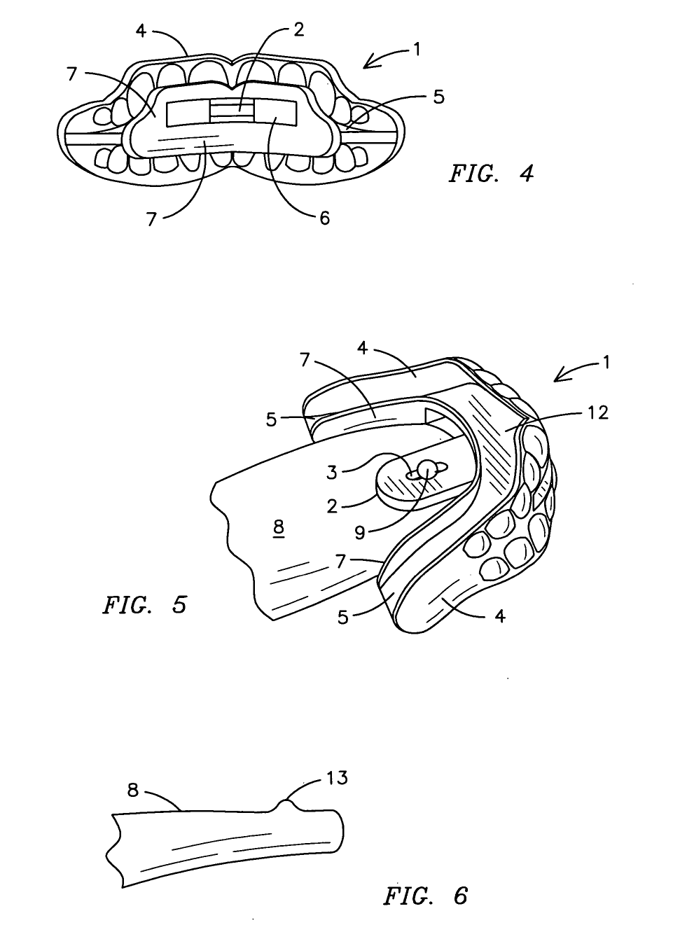

Next, FIG. 4 shows a rear view of the present invention. The first walls 4 of the tongue retention device 1 are substantially taller than the second walls 7 of the tongue retention device 1 so as to provide a comfortable fit in a person's mouth.

FIG. 5 shows a perspective view of the present invention in use. The attachment device 9 is secured through the aperture 3 of the tab 2 such that the attachment device 9 does not readily slip into or fall out of the aperture 3.

Finally, FIG. 6 shows a side view of a second embodiment of an attachment device 9 for use in conjunction with the present invention. Rather than the attachment device 9 being a tongue piercing as shown in FIGS. 3 and 5, the attachment device 9 may also be in the form of an implant 13 that is embedded into and non-removable from the tongue 8. Because the implant 13 is made of a biocompatible material and is permanently embedded in the tongue 8, rather than being removably located through the tongue 8 as when using a piercing, the implant 13 attachment device 9 is more sanitary and easier to use. In addition, a person need not worry about a piercing hole closing up if he or she is using the implant 13.

As described above, the use of the present invention will maintain a person's tongue in a forward position while asleep.

It is to be understood that while a preferred embodiment of the invention is illustrated, it is not to be limited to the specific form or arrangement of parts herein described and shown. It will be apparent to those skilled in the art that various changes may be made without departing from the scope of the invention and the invention is not be considered limited to what is shown and described in the specification and drawings.

Having thus described my invention, I claim:

1. A tongue retention device comprising:
an anchoring piece having a first wall and a second wall;
a channel located between said first wall and said second wall for the acceptance of at least one tooth;
a tab secured to said anchoring piece;
at least one aperture located in said tab for the acceptance of an attachment device; and
an attachment device located in a person's tongue.

2. The tongue retention device of claim 1 wherein:
said tongue retention device is plastic.

3. The tongue retention device of claim 1 wherein:
said tongue retention device is rubber.

4. The tongue retention device of claim 1 wherein:
said at least one aperture located in said tab is oval in shape so as to allow for slight tongue movement.

5. The tongue retention device of claim 1 wherein:
said attachment device is a tongue-piercing.

6. The tongue retention device of claim 1 wherein:
said attachment device is an implant.

7. A tongue retention device comprising:
an anchoring piece having a first wall and a second wall;
a channel located between said first wall and said second wall for the acceptance of at least one tooth;
a tab secured to said anchoring piece;
at least one aperture located in said tab sized for the acceptance of an attachment device;
an attachment device located in a person's tongue;
said attachment device is a tongue-piercing; and
said tongue retention device is rubber.

8. A method for using a tongue retention device comprising an anchoring piece having a first wall and a second wall; a channel located between said first wall and said second wall for the acceptance of at least one tooth; a tab secured to said anchoring piece; at least one aperture located in said tab for the acceptance of an attachment device; and an attachment device located in a tongue, said method comprising the steps of:
a. inserting said tongue retention device into a mouth;
b. inserting said attachment device into said at least one aperture; and
c. positioning at least one tooth within said channel.
